SEQUENCE LISTING (16 sequences)

SEQ ID NO: 1 - DNA, 20 bp, Artificial Sequence
  Description of Artificial Sequence primer
accacagtccatgccatcac

SEQ ID NO: 2 - DNA, 20 bp, Artificial Sequence
  Description of Artificial Sequence primer
tccaccaccctgttgctgta

SEQ ID NO: 3 - DNA, 20 bp, Artificial Sequence
  Description of Artificial Sequence primer
ttttgggacccgaactttcc

SEQ ID NO: 4 - DNA, 23 bp, Artificial Sequence
  Description of Artificial Sequence primer
ctcctggtatcctttagcaactc

SEQ ID NO: 5 - DNA, 24 bp, Artificial Sequence
  Description of Artificial Sequence primer
gcacaccagtccaacagcaccaat

SEQ ID NO: 6 - DNA, 25 bp, Artificial Sequence
  Description of Artificial Sequence primer
ccgaagttgacagtgaaggcttctg

SEQ ID NO: 7 - DNA, 20 bp, Artificial Sequence
  Description of Artificial Sequence primer
ggtgttgattgcctttgctc

SEQ ID NO: 8 - DNA, 20 bp, Artificial Sequence
  Description of Artificial Sequence primer
cccttcatcccgaagttcat

SEQ ID NO: 9 - DNA, 20 bp, Artificial Sequence
  Description of Artificial Sequence primer
ctcccaaggcacagagacac

SEQ ID NO: 10 - DNA, 20 bp, Artificial Sequence
  Description of Artificial Sequence primer
ggagccgtagtgagcagttc

SEQ ID NO: 11 - DNA, 20 bp, Artificial Sequence
  Description of Artificial Sequence primer
cgaaaaagccacacattcct

SEQ ID NO: 12 - DNA, 20 bp, Artificial Sequence
  Description of Artificial Sequence primer
cacggtacttgtccacgatg

SEQ ID NO: 13 - DNA, 20 bp, Artificial Sequence
  Description of Artificial Sequence primer
aagagatccgcacgcagtat

SEQ ID NO: 14 - DNA, 20 bp, Artificial Sequence
  Description of Artificial Sequence primer
gtaggtggcgatctcgatgt

SEQ ID NO: 15 - DNA, 20 bp, Artificial Sequence
  Description of Artificial Sequence primer
tgcgggctactgaaaagttc

SEQ ID NO: 16 - DNA, 20 bp, Artificial Sequence
  Description of Artificial Sequence primer
ggggagggaagttgggctca

(19) United States
(12) Patent Application Publication
(10) Pub. No.: US 2016/0222347 A1
(43) Pub. Date: Aug. 4, 2016

(54) METHOD FOR INDUCING DIFFERENTIATION OF INDUCED PLURIPOTENT STEM CELLS AND METHOD FOR SELECTING THE SAME

(71) Applicants: Kazuhito SATOMURA, Tokushima-shi, Tokushima (JP); KANEKA CORPORATION, Osaka-shi, Osaka (JP)

(72) Inventor: Kazuhito SATOMURA, Tokushima-shi (JP)

(73) Assignee: KANEKA CORPORATION, Osaka-shi, Osaka (JP)

(21) Appl. No.: 15/021,615

(22) PCT Filed: Sep. 5, 2014
(86) PCT No.: PCT/JP2014/073511

§ 371 Date: Mar. 11, 2016

(30) Foreign Application Priority Data
Sep. 12, 2013 (JP) 2013-189221

(51) Int. Cl. C12N 5/071; C12N 5/077; C12N 5/079

(52) U.S. Cl. CPC C12N 5/067; C12N 5/0618; C12N 5/0652; C12N 2506/45

(57) ABSTRACT

It is an object of the present invention to provide a method for highly efficiently inducing differentiation of iPS cells into cells having functions of interest, and a method for selecting iPS cells having high differentiation induction efficiency for differentiation of the iPS cells into cells of interest from among the produced iPS cells. The present invention relates to a method for inducing differentiation of induced pluripotent stem cells, which comprises culturing induced pluripotent stem cells on a structure comprising cells and/or a component derived from the cells, wherein the cells in the structure and the differentiation-induced cells are the same type of cell.

### TECHNICAL FIELD

The present invention relates to a method for inducing differentiation of induced pluripotent stem cells, a method for selecting induced pluripotent stem cells, and a kit for selecting induced pluripotent stem cells.

### BACKGROUND ART

Stem cells play an important role in regenerative medicine. Stem cells that are known to have totipotency include embryonic stem cells (ES cells), embryonic carcinoma stem cells (EC cells), embryonic germ stem cells (EG cells), nuclear transfer ES cells, somatic cell-derived ES cells (ntES cells), and induced pluripotent stem cells (iPS cells). Stem cells that are known to have pluripotency include somatic stem cells, tissue stem cells, and adult stem cells. Among the aforementioned cells, induced pluripotent stem cells (iPS cells) have totipotency and are artificially produced from somatic cells. Thus, since iPS cells do not have either ethical problems regarding destruction of embryos or eggs, or problems regarding incompatibility in transplantation, it is anticipated that the iPS cells will be applied to regenerative medicine.

With regard to a method for inducing differentiation of induced pluripotent stem cells (iPS cells) into desired cells, various types of methods have been reported. For example, induction of differentiation of iPS cells into pancreatic cells, hepatic cells, cardiomyocytes, blood cells, germ cells, nerve cells, and the like has been reported. However, for practical use of regenerative medicine, in which induced pluripotent stem cells (iPS cells) are used, it is necessary to establish a method for highly efficiently inducing differentiation of iPS cells into cells having functions of interest.

Patent Literature 1 describes a method for inducing differentiation of stem cells into a certain cell lineage, which comprises culturing stem cells in vitro in the presence of a tissue sample and/or an extracellular medium of the tissue sample under conditions for inducing differentiation of the stem cells into the certain cell lineage, wherein the differentiated stem cells have the same cell type as that of the tissue sample. The stem cells used in Patent Literature 1 are embryonic stem cells (ES cells), and Patent Literature 1 contains no descriptions regarding induced pluripotent stem cells (iPS cells).

### PRIOR ART LITERATURES

### Patent Literature

Patent Literature 1: JP Patent Publication (Kohyo) No. 2005-520516 A

### SUMMARY OF INVENTION

### Object to be Solved by the Invention

A problem regarding the practical use of regenerative medicine, in which induced pluripotent stem cells (iPS cells) are used, is considered to be the establishment of a technique of highly efficiently inducing differentiation of undifferentiated iPS cells into cells having functions of interest. In addition, another problem is considered to be the establishment of a method for evaluating and/or controlling the quality of iPS cells. That is to say, it is an object of the present invention to provide a method for highly efficiently inducing differentiation of iPS cells into cells having functions of interest, and a method for selecting iPS cells having high differentiation induction efficiency for differentiation of the produced iPS cells into cells of interest. It is another object of the present invention to provide a kit for selecting iPS cells, which is used for the aforementioned methods.

### Means for Solving the Object

As a result of intensive studies directed towards achieving the aforementioned objects, the present inventors have found that differentiation of iPS cells into cells of interest can be achieved by culturing the iPS cells on a frozen section of tissue and/or organ that is to be regenerated, thereby completing the present invention.
(1) A method for inducing differentiation of induced pluripotent stem cells, which comprises culturing induced pluripotent stem cells on a structure comprising cells and/or a component derived from the cells, wherein the cells in the structure and the differentiation-induced cells are the same type of cell.
(2) The method according to (1), wherein the structure comprising cells and/or a component derived from the cells is a sheet-like structure.
(3) The method according to (1) or (2), wherein the structure comprising cells and/or a component derived from the cells is a culture substrate coated with a component derived from biological tissues, organ sections or cells.
(4) The method according to any one of (1) to (3), wherein the cells are cells of the liver, brain or spinal cord.
(5) A method for producing differentiation-induced cells, which comprises culturing induced pluripotent stem cells on a structure comprising cells and/or a component derived from the cells, wherein the cells in the structure and the differentiation-induced cells are the same type of cell.
(6) The method according to (5), wherein the structure comprising cells and/or a component derived from the cells is a sheet-like structure.
(7) The method according to (6) or (7), wherein the structure comprising cells and/or a component derived from the cells is a culture substrate coated with a component derived from biological tissues, organ sections or cells.
(8) The method according to any one of (5) to (7), wherein the cells are cells of the liver, brain or spinal cord.
(9) A method for selecting induced pluripotent stem cells, which comprises culturing induced pluripotent stem cells on a structure comprising cells and/or a component derived from the cells, so as to induce differentiation of the induced pluripotent stem cells, and selecting induced pluripotent stem cells having high differentiation induction efficiency, wherein the cells in the structure and the differentiation-induced cells are the same type of cell.
(10) The method according to (9), wherein the structure comprising cells and/or a component derived from the cells is a sheet-like structure.
(11) The method according to (9) or (10), wherein the structure comprising cells and/or a component derived from the cells is a culture substrate coated with a component derived from biological tissues, organ sections or cells.
(12) The method according to any one of (9) to (11), wherein the cells are cells of the liver, brain or spinal cord.
(13) A kit for selecting induced pluripotent stem cells, which comprises at least a structure comprising cells and/or a component derived from the cells.
(14) The kit according to (13), wherein the structure comprising cells and/or a component derived from the cells is a sheet-like structure.
(15) The kit according to (13) or (14), wherein the structure comprising cells and/or a component derived from the cells is a culture substrate coated with a component derived from biological tissues, organ sections or cells.
(16) The kit according to any one of (13) to (15), wherein the cells are cells of the liver, brain or spinal cord.

### Advantageous Effects of Invention

According to the present invention, it is possible to highly efficiently induce differentiation of iPS cells into cells having functions of interest. Moreover, according to the present invention, it is also possible to select iPS cells having high differentiation induction efficiency for differentiation of the iPS cells into cells of interest from among the produced iPS cells.

### BRIEF DESCRIPTION OF DRAWINGS

FIG. 1 shows microscopic images of iPS cells cultured on a cover glass (control), on a normal liver section, or on a hepatitis liver section (Day 2 and Day 8 of the culture).

FIG. 2 shows microscopic images of iPS cells cultured on a cover glass (control) or on a normal liver section (Day 3 and Day 9 of the culture).

FIG. 3 shows microscopic images of iPS cells cultured on a brain section or on a spinal cord section (Day 3 and Day 9 of the culture).

FIG. 4 shows the results obtained by analyzing the expression of hepatic cell-related genes (AFP, AAT, and ALB) by RT-PCR.

FIG. 5 shows the results obtained by analyzing the expression of nerve cell-related genes (Nestin, MBP, CNPase, and GFAP) by RT-PCR

FIG. 6 shows the results obtained by immunocytochemically analyzing the expression of AFP.

FIG. 7 shows the results obtained by measuring the ratio of AFP-positive cells in nucleated cells.

FIG. 8 shows the results obtained by immunocytochemically analyzing the expression of AAT.

FIG. 9 shows the results obtained by measuring the ratio of AAT-positive cells in nucleated cells.

FIG. 10 shows the results obtained by immunocytochemically analyzing the expression of GFAP.

FIG. 11 shows the results obtained by measuring the ratio of GFAP-positive cells in nucleated cells.

FIG. 12 shows the results obtained by immunocytochemically analyzing the expression of CNPase.

FIG. 13 shows the results obtained by immunocytochemically analyzing the expression of CNPase.

FIG. 14 shows the results obtained by measuring the ratio of CNPase-positive cells in nucleated cells.

### EMBODIMENTS FOR CARRYING OUT THE INVENTION

Hereinafter, the present invention will be described more in detail.

### (1) Method for Inducing Differentiation of Induced Pluripotent Stem Cells, and Method for Producing Differentiation-Induced Cells

The present invention relates to a method for inducing differentiation of induced pluripotent stem cells or a method for producing the differentiation-induced cells, each of which comprises culturing induced pluripotent stem cells on a structure comprising cells and/or a component derived from the cells, wherein, in particular, the cells in the structure and the differentiation-induced cells are of the same cell type. Whether or not the cells in the structure and the differentiation-induced cells are of the same cell type can be determined based on, for example, the fact that the same markers are expressed in the two types of cells.

Examples of a cell marker for hepatic cells include, but are not limited to, α-fetoprotein (AFP), α-1 antitrypsin (AAT), albumin (ALB), tyrosine aminotransferase (TAT), tryptophan 2,3 dioxygenase (TDO2), and cytochrome P450.

Examples of a marker for nerve cells include, but are not limited to, nestin, myelin basic protein (MBP), cyclic nucleotide phosphodiesterase (CNPase), glial fibrillary acidic protein (GFAP), and neurofilament.

Examples of a marker for osteoblasts include, but are not limited to, alkaline phosphatase (ALP), osteopontin, and osteocalcin.

Examples of a marker for pancreatic cells include, but are not limited to, Pdx1, amylase, and carboxypeptidase.

Examples of a marker for chondrocytes include, but are not limited to, Sox9, type II collagen, and aggrecan.

Examples of a marker for cardiomyocytes include, but are not limited to, cardiac troponin I (cTnI), α-myosin heavy chain (α-MHC), α-cardiac actin, and homeobox protein Nkx-2.5.

The form of the “structure comprising cells and/or a component derived from the cells” used in the present invention is not particularly limited. Taking into consideration the fact that induced pluripotent stem cells (iPS cells) are cultured on this structure, the structure is preferably a sheet-like structure. As an example of a sheet-like structure comprising cells and/or a component derived from the cells, a culture substrate coated with a component derived from biological tissues, organ sections or cells can be used. The thickness of the sheet-like structure comprising cells (preferably, a section of biological tissue or organ) is not particularly limited. It is generally about 1 to 100 μm, preferably about 2 to 50 μm, and more preferably about 2 to 20 μm.

Such a structure comprising a component derived from cells can be obtained by purifying the component derived from cells using a nucleic acid, micro RNA, an antibody or a compound having an interaction with the component derived from cells, allowing the purified component derived from cells to adhere onto a culture substrate, and then coating it with an enzyme, a surfactant or the like. Moreover, the coated culture substrate is further dried, so as to provide a long-term storable culture substrate.

The form of the “culture substrate” used in the present invention is not particularly limited. It is preferably a film, a plate or a cover glass.

A tissue or organ section can be preferably collected from a mammal (preferably, a mouse, a human, etc.). In the case of using a biological tissue or organ section, the type of the tissue or organ is not particularly limited, and a tissue or organ section comprising cells of the same cell type as that of differentiated cells which are differentiated from induced pluripotent stem cells (iPS cells), may be used. Examples of such a tissue or organ include, but are not limited to, liver, brain, spinal cord, heart, respiratory organ, reproductive organ, kidney, pancreas, skin, muscle, and skeletal organ. For example, when iPS cells are induced to differentiate into hepatic cells, a liver section may be used, and when iPS cells are induced to differentiate into nerve cells, a section comprising nerve cells (for example, a brain section, a spinal cord section, etc.) may be used.

Examples of a component derived from cells include a nucleic acid (DNA or RNA, and in particular, micro RNA, etc.) and a protein. In particular, a nucleic acid (DNA or RNA, and in particular, micro RNA) or a protein, which is specifically expressed in the aforementioned cells, is preferable.

According to the method of the present invention, induced pluripotent stem cells are induced to differentiate into certain cell lineage, and preferably into a cell lineage, such as liver, nerve, lung, prostate gland, pancreas, mammary gland, kidney, bowel, skeleton, blood vessel, hematopoietic organ, cardiac muscle, or skeletal muscle.

Induced pluripotent stem cells have been established for the first time by introducing four factors Oct3/4, Sox2, Klf4 and c-Myc into mouse fibroblasts, and the established cells have been named as “iPS cells” (Takahashi K, Yamanaka S., Cell, (2006) 126: 663-676). Subsequently, human iPS cells have also been established using the same four factors as described above (Takahashi K, Yamanaka S., et al., Cell, (2007) 131: 861-872.). Moreover, a method of using three factors excluding c-Myc has also been reported (Nakagawa M, Yamanaka S., et al., Nature Biotechnology, (2008) 26, 101-106). Furthermore, it has also been reported that four genes OCT3/4, SOX2, NANOG and LIN28 are introduced into human fibroblasts, so as to establish induced pluripotent stem cells (Yu J., Thomson J A, et al., Science (2007) 318: 1917-1920.). Further, it has also been reported that six genes OCT3/4, SOX2, KLF4, C-MYC, hTERT and SV40 large T are introduced into skin cells to establish induced pluripotent stem cells (Park I H, Daley G Q, et al., Nature (2007) 451: 141-146), and that Oct3/4, Sox2, Klf4, c-Myc, and the like are introduced, not into somatic cells, but into undifferentiated stem cells that are present in postnatal tissues, so as to establish induced pluripotent stem cells (JP Patent Publication (Kokai) No. 2008-307007 A).

As described above, induced pluripotent stem cells (iPS cells) mean cells having multipotency and self-replication ability, which are induced by reprogramming somatic cells or undifferentiated stem cells. The origin of somatic cells is not limited, and the somatic cells may be derived from any one of an embryo, a fetus, and an adult. An animal species, from which somatic cells are derived, is not particularly limited, either. The animal species is preferably a mammal, and more preferably a human, a mouse, or the like. Examples of the somatic cells include, but are not limited to, fibroblasts, epithelial cells, hepatic cells, and blood cells. The method for producing induced pluripotent stem cells used in the present invention is not particularly limited, and a factor to be introduced, an introduction method, and the like are not particularly limited, either. In addition to the above-mentioned publications, examples of known publications regarding induced pluripotent stem cells include JP Patent Publication (Kokai) No. 2008-283972 A, US 2008-2336610, US 2009-047263, WO 2007/069666, WO2008/118220, WO 2008/124133, WO 2008/151058, WO 2009/057831, WO 2009/006997, and WO 2009/007852.

In the present invention, after iPS cells have been cultured on feeder cells according to an ordinary method, the feeder cells are removed, and iPS cells are recovered. The recovered iPS cells are suspended in a suitable medium (for example, a Dulbecco's modified Eagle's medium (DMEM) containing fetal bovine serum, etc.). Subsequently, there is prepared a culture cover glass, on which a structure comprising cells (preferably, a biological tissue or organ section) is placed, and the suspension of iPS cells may be added to this cover glass. After addition of the suspension, the cover glass is left at rest, so that floating iPS cells are allowed to adhere to the structure comprising cells. Thereafter, a suitable medium (DMEM containing 5% FBS, etc.) is added thereto, and a culture may be then carried out according to an ordinary method.

Conditions for culturing induced pluripotent stem cells on a structure comprising cells and/or a component derived from the cells are not particularly limited, as long as differentiation of induced pluripotent stem cells can be induced under the conditions. As a medium, for example, a Dulbecco's modified Eagle's medium (DMEM) containing fetal bovine serum, or a medium used for primate ES cells (ReproCELL Inc.) can be used. Various types of growth factors, cytokines, differentiation-inducing factors and the like, which are used to induce differentiation of induced pluripotent stem cells, may be added to the medium before performing the culture. However, in the present invention, by performing a culture on a structure comprising cells, induction of differentiation of induced pluripotent stem cells can be achieved without adding the aforementioned growth factors or differentiation-inducing factors. Examples of various types of growth factors, cytokines or differentiation-inducing factors, which are used to induce differentiation of induced pluripotent stem cells, include, but are not limited to, activin, bFGF, noggin, nicotinamide, retinoic acid, EGF, and glucocorticoid.

### (2) Method for Selecting Induced Pluripotent Stem Cells

According to the present invention, induced pluripotent stem cells are cultured on a structure comprising cells and/or a component derived from the cells, so as to induce differentiation of the induced pluripotent stem cells, so that induced pluripotent stem cells having high differentiation induction efficiency can be selected. As described above, at present, induced pluripotent stem cells are produced by various methods. It is anticipated that the produced induced pluripotent stem cells are induced to differentiate into desired cells, and they are then applied to regenerative medicine and the like. However, in general, the produced induced pluripotent stem cells comprise both cells having high differentiation induction efficiency for differentiating into desired cells and cells having low differentiation efficiency. Thus, it has been desired to develop a technique of selecting induced pluripotent stem cells having high differentiation induction efficiency for differentiation of the produced induced pluripotent stem cells into desired cells from.

According to the present invention, by culturing induced pluripotent stem cells on a structure comprising cells and/or a component derived from the cells, differentiation of the induced pluripotent stem cells are induced, and the induced pluripotent stem cells having high differentiation induction efficiency for differentiating into desired cells can be selected. The thus selected induced pluripotent stem cells having high differentiation induction efficiency are preserved as a stock, and at the time of need, the preserved cells can be induced to differentiate into desired cells and can be then used in the site of regenerative medicine.

Moreover, the structure comprising cells and/or a component derived from the cells, such as a biological tissue or organ section, can be provided as a kit for selecting induced pluripotent stem cells. The aforementioned kit may also comprise, as appropriate, a medium for culturing induced pluripotent stem cells, a cover glass on which the structure comprising cells is established, and the like, in addition to the structure comprising cells and/or a component derived from the cells.

The present invention will be described in detail in the following examples. However, these examples are not intended to limit the scope of the present invention.

### EXAMPLES

### (1) Production of Frozen Section

Normal liver, brain and spinal cord were excised from a 6-week-old male ICR mouse. In addition, 1 mg/kg carbon tetrachloride that had been mixed with olive oil at a mixing ratio of 1:4 was intraperitoneally administered to ICR mice, so that drug-induced hepatitis was artificially developed in the mice. From these ICR mice, on 1, 2, 3 and 5 days after administration of carbon tetrachloride, hepatitis liver was excised.

Each organ was embedded and mounted in Tissue-Tek^{R} (Sakura Finethechnical) that was an OCT compound, and it was then immersed in liquid nitrogen to produce a block for production of a frozen section. From each of these blocks, a frozen section with a thickness of 6 μm was produced, and was then placed on a circular culture cover glass (poly L lysine-coated type, Matsunami Glass Ind., Ltd.). After drying, it was washed with a phosphate buffer (Phosphate Buffered Saline; hereinafter abbreviated as “PBS”; pH 7.4) twice to wash the OCT compound off, and the resultant was then used for the culture of iPS cells.

### (2) Culture of iPS Cells and Induction of Differentiation Thereof

iPS cells were cultured according to an ordinary method, and feeder cells were then removed using an iPS/ES cell releasing solution (CTK solution, ReproCELL Inc.), followed by washing with PBS. Thereafter, the iPS cells were recovered and were then suspended in a Dulbecco's modified Eagle's medium (hereinafter abbreviated as “DMEM”, SIGMA) containing 5% fetal bovine serum (hereinafter abbreviated as “FBS”, Biological Industries). The above-described circular culture cover glass, on which a frozen section had been placed, was placed on a dish with a diameter of 35 min (non-coated type, Matsunami Glass hid., Ltd.), and the iPS cell suspension was then inoculated thereon. The suspending iPS cells were allowed to adhere to the frozen section by leaving the cells at rest for 1 day after completion of the inoculation, and 2 ml of DMEM containing 5% FBS was then added thereto to carry out a culture. Thereafter, the medium was exchanged with a fresh one once three days, and the culture was carried out for a total of 9 days.

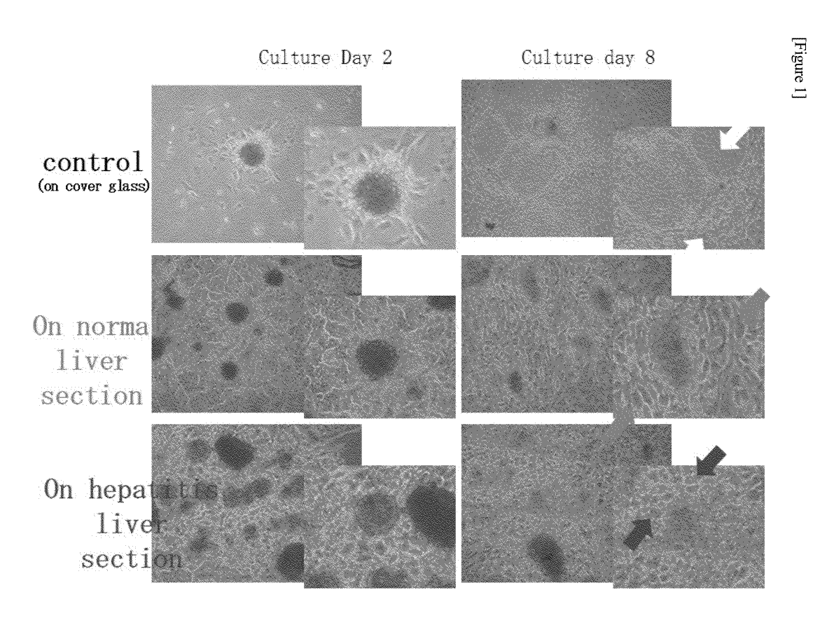

The microscopic images (Day 2 and Day 8 of the culture) of iPS cells cultured on a cover glass (control), on a normal liver section, and on a hepatitis liver section are shown in FIG. 1. In all cases of the control, the normal liver, and the hepatitis liver, it was observed that a colony of the inoculated iPS cells extended and the cells were dispersed. Moreover, a change in the shape of cell was also observed, and in the case of the control, when compared with Day 2 of the culture, the extended iPS cells on Day 8 of the culture had various shapes, as shown with the arrow, and no uniformity was found. On the other hand, in the cases of the normal liver and the hepatitis liver, the extended cells tended to have a relatively large and polygonal shape, as shown with the arrow.

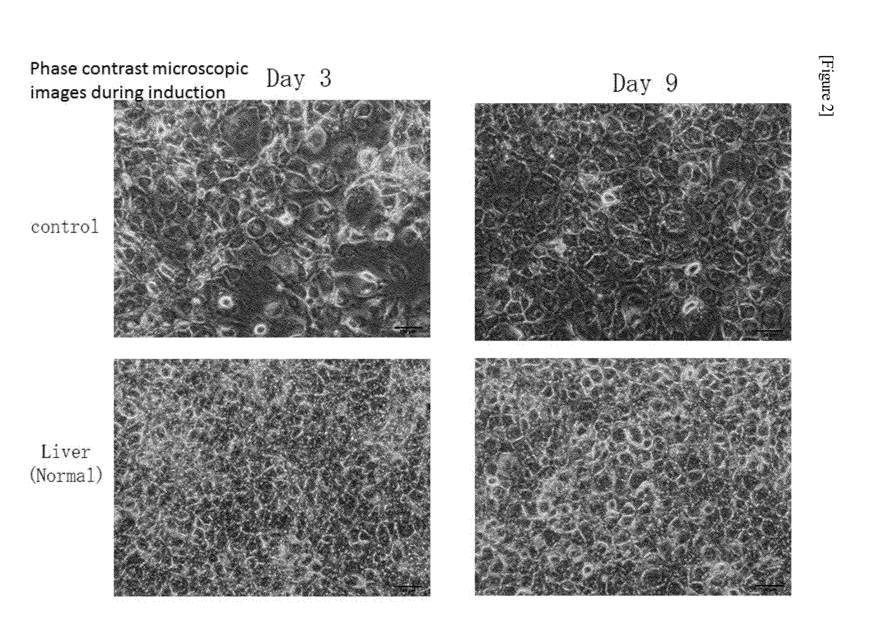

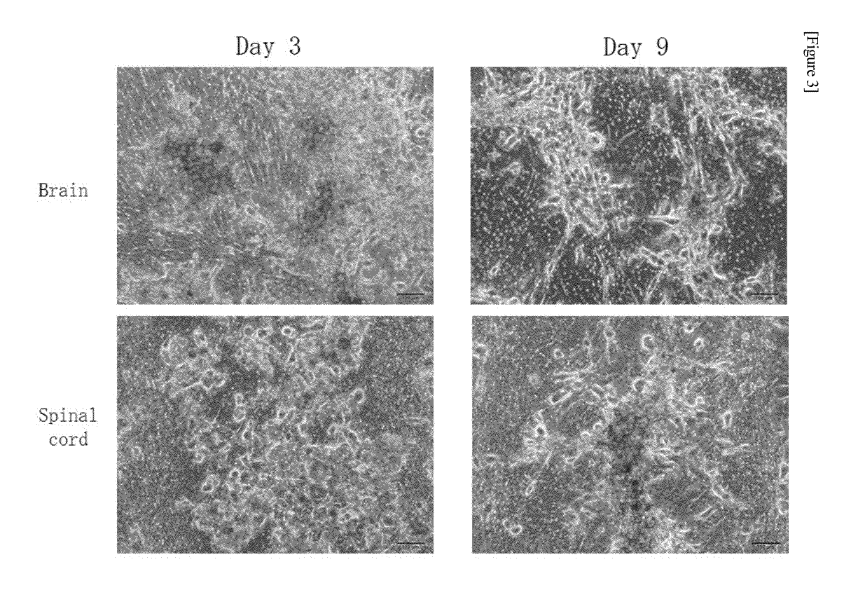

Furthermore, the microscopic images (Day 3 and Day 9 of the culture) of iPS cells cultured on a cover glass (control) and on a normal liver section are shown in FIG. 2, and the microscopic images (Day 3 and Day 9 of the culture) of iPS cells cultured on a brain section and on a spinal cord section are shown in FIG. 3.

### (3) Confirmation of Gene Expression

In order to confirm the differentiation state of iPS cells into hepatic cells or nerve cells, the expression of mRNA of various differentiation markers for nerve cells and hepatic cells was analyzed using a semi-quantitative Reverse Transcriptase-Polymerase Chain Reaction method (hereinafter abbreviated as “RT-PCR method”). Total RNA was recovered from iPS cells, which had been cultured on the frozen sections of liver, brain and spinal cord, using TRIzol reagent (Invitrogen Corp., Carlsbad, Calif., USA). 1 μg of the obtained total RNA was treated at room temperature with 100 units/ml deoxyribonuclease I (hereinafter abbreviated as “DNase I”), and 1 ml of 25 mM EDTA was then added to the RNA. The obtained mixture was treated at 65° C. for 5 minutes, so as to inactivate DNase I. A random hexamer primer (Invitrogen) was added to the reaction mixture, and a reverse transcription reaction was then carried out using reverse transcriptase SUPERSCRIPT III Preamplification System (Invitrogen) under conditions of 50° C. and 50 minutes. The reaction mixture was further treated with ribonuclease H at 37° C. for 20 minutes, so as to obtain cDNA. Using primers specific to each human gene and PCR MasterMix Kit (Thermo, Rockford, USA), PCR was carried out, and the expression of the mRNA of each gene was then studied. The studied differentiation markers, primers and PCR conditions are shown in Table 1. The PCR product was electrophoresed using 2% agarose gel, was then stained with ethidium bromide, and was then visualized using the UV imaging apparatus FAS-III (TOYOBO Co., Ltd., Osaka).

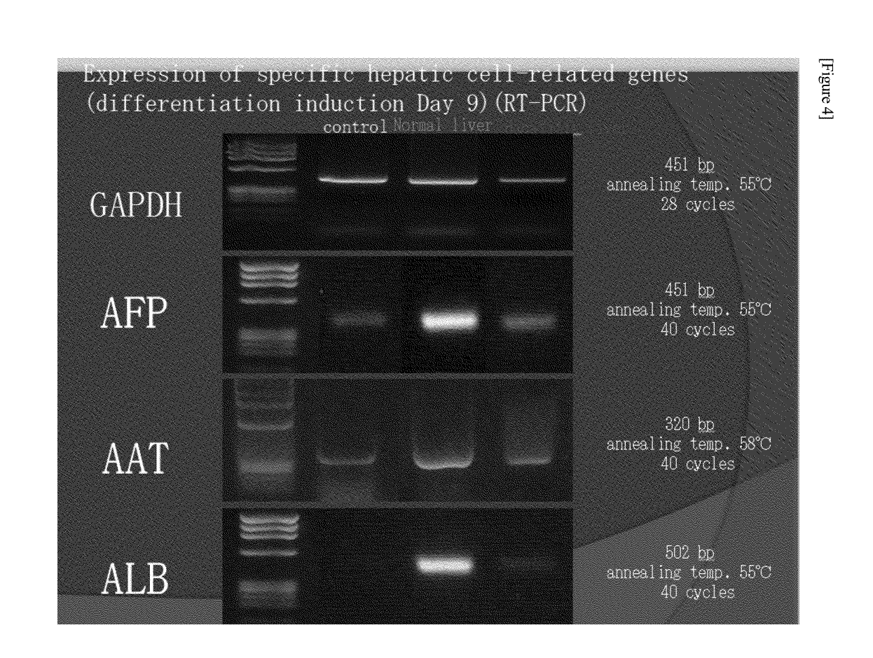

The results obtained by analyzing the expression of hepatic cell-related genes (AFP, AAT, and ALB) are shown in FIG. 4.

The expression of AFP was observed in the control group, the normal liver group, and the hepatitis liver group. The expression of AFP tended to be strong in the hepatitis liver group, in comparison to in the control group, and the expression was further increased in the normal liver group. The expression of AAT was also observed in the control group, the normal liver group, and the hepatitis liver group. The expression of AAT was strong in the normal liver group, in comparison to in the control group. On the other hand, the expression of ALB was not observed in the control group, but the expression was confirmed in the normal liver group and the hepatitis liver group. The expression of ALB was strong in the normal liver group, in comparison to in the hepatitis liver group. From these results, it was confirmed that differentiation of iPS cells into specific hepatic cells was induced by culturing the iPS cells on the frozen section of normal liver and hepatitis liver.

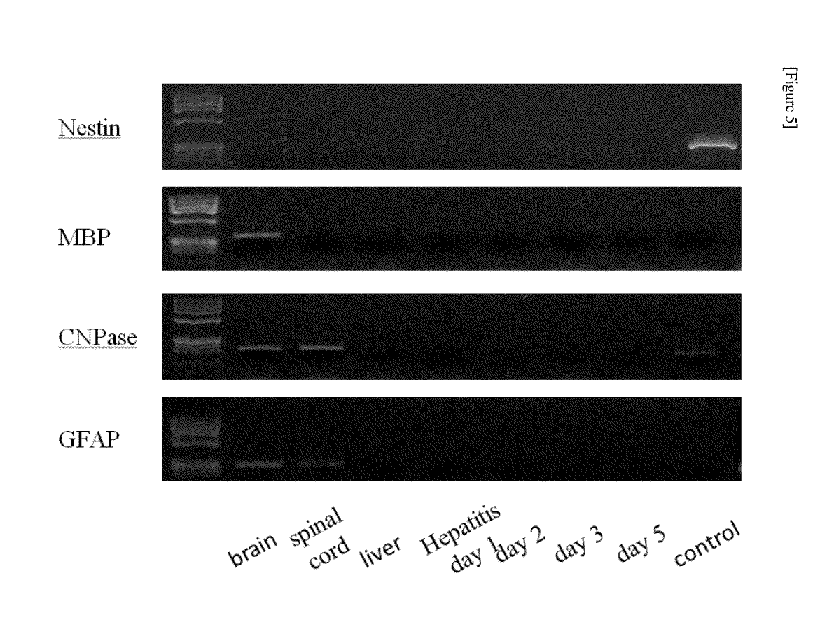

Likewise, the results obtained by analyzing the expression of nerve cell-related genes (Nestin, MBP, CNPase, and GFAP) are shown in FIG. 5.

TABLE 1

| | | | Size | Annealing | Number |
|---|---|---|---|---|---|
| | Sequence | | (bp) | temperature | of cycles |
| GAPDH | F: | 5′-acc aca gtc cat gcc atc ac-3′ | 451 | 55 | 30 |
| | | (SEQ ID NO: 1) | | | |
| | R: | 5′-tcc acc acc ctg ttg ctg ta-3′ | | | |
| | | (SEQ ID NO: 2) | | | |
| AFP | F: | 5′-ttt tgg gac ccg aac ttt cc-3′ | 451 | 55 | 40 |
| | | (SEQ ID NO: 3) | | | |
| | R: | 5′-ctc ctg gta tcc ttt agc aac tc-3′ | | | |
| | | (SEQ ID NO: 4) | | | |
| AAT | F: | 5′-gca cac cag tcc aac agc acc aat-3′ | 320 | 58 | 40 |
| | | (SEQ ID NO: 5) | | | |
| | R: | 5′-ccg aag ttg aca gtg aag gct tct g-3′ | | | |
| | | (SEQ ID NO: 6) | | | |
| ALB | F: | 5′-ggt gtt gat tgc ctt tgc tc-3′ | 502 | 55 | 40 |
| | | (SEQ ID NO: 7) | | | |
| | R: | 5′-ccc ttc atc ccg aag ttc at-3′ | | | |
| | | (SEQ ID NO: 8) | | | |
| MBP | F: | 5′-ctc cca agg cac aga gac ac-3′ | 207 | 58 | 40 |
| | | (SEQ ID NO: 9) | | | |
| | R: | 5′-gga gcc gta gtg agc agt tc-3′ | | | |
| | | (SEQ ID NO: 10) | | | |
| CNPase | F: | 5′-cga aaa agc cac aca ttc ct-3′ | 208 | 58 | 40 |
| | | (SEQ ID NO: 11) | | | |
| | R: | 5′-cac ggt act tgt cca cga tg-3′ | | | |
| | | (SEQ ID NO: 12) | | | |
| GFAP | F: | 5′-aag aga tcc gca cgc agt at-3′ | 392 | 58 | 40 |
| | | (SEQ ID NO: 13) | | | |
| | R: | 5′-gta ggt ggc gat ctc gat gt-3′ | | | |
| | | (SEQ ID NO: 14) | | | |
| NES | F: | 5′-tgc ggg cta ctg aaa agt tc-3′ | 311 | 58 | 40 |
| | | (SEQ ID NO: 15) | | | |
| | R: | 5′-ggg gag gga agt tgg gct ca-3′ | | | |
| | | (SEQ ID NO: 16) | | | |

GAPDH: Glyceraldehyde-3-phosphate dehydrogenase
AFP: α-Fetoprotein
AAT: α1-Antitrypsin
ALB: Albumin
MBP: Myelin basic protein
CNPase: 2′,3′-Cyclic nucleotide 3′-phosphodiesterase
GFAP: Glial fibrillary acidic protein
NES: Nestin

### (4) Confirmation of Protein Expression

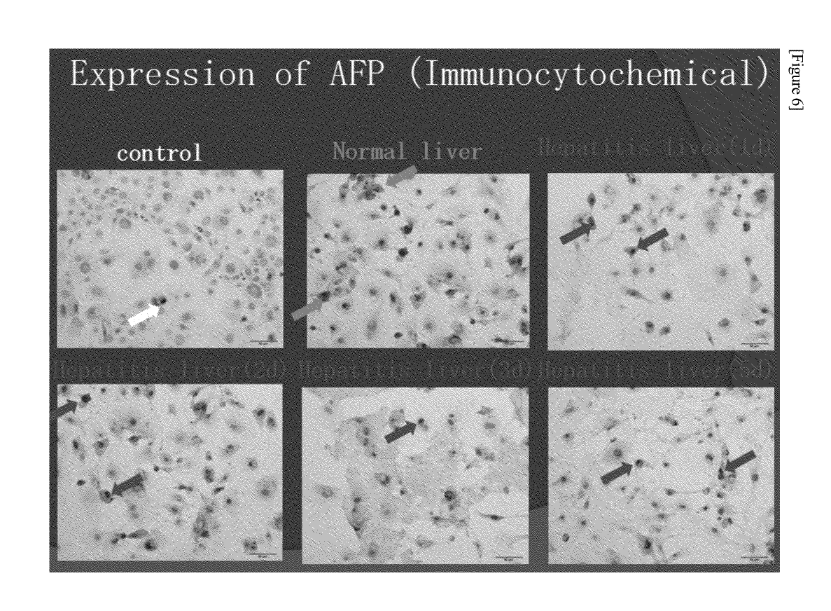

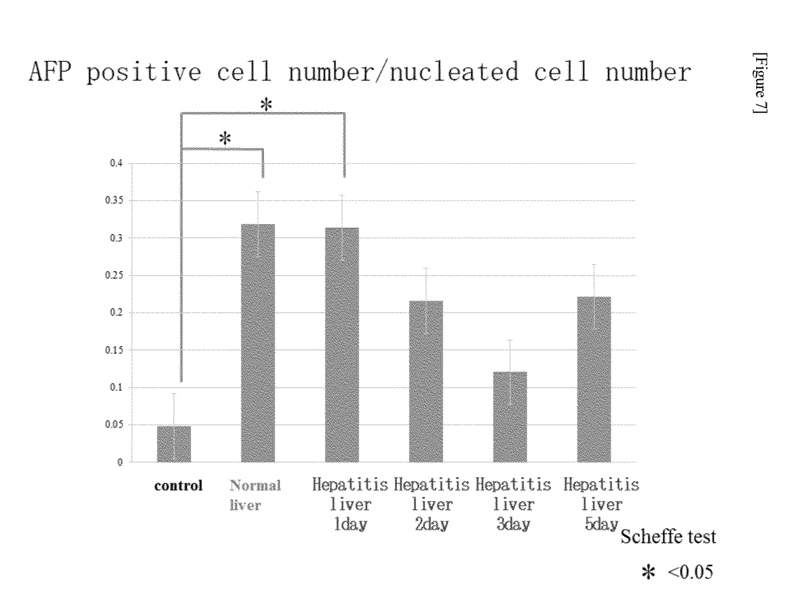

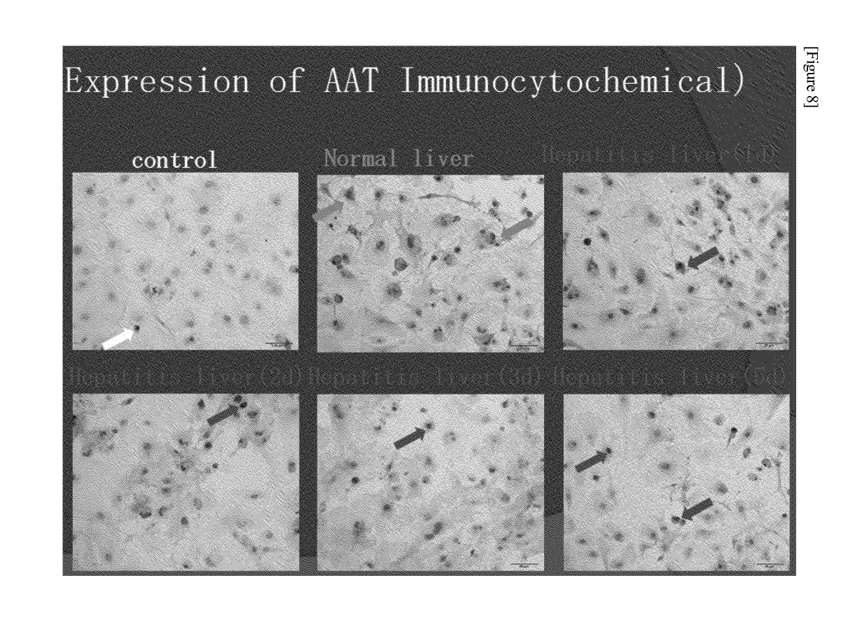

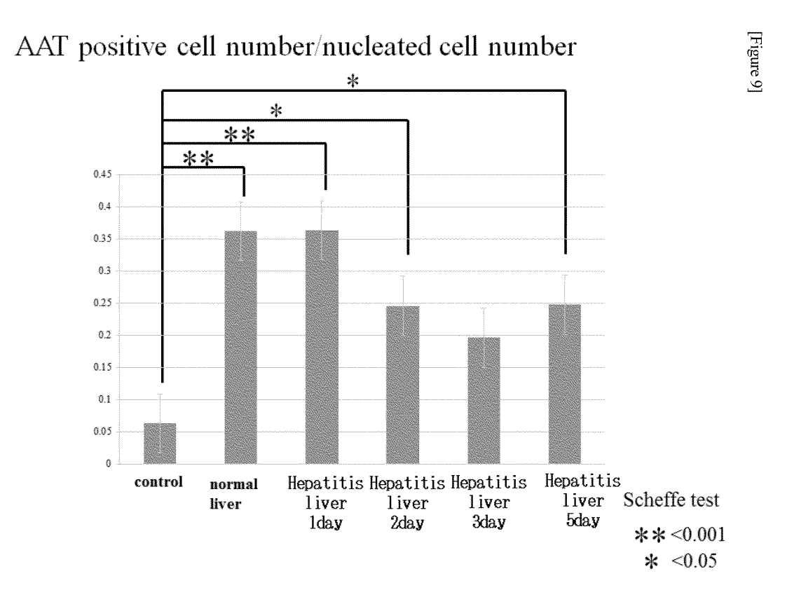

iPS cells cultured on various types of frozen sections were washed with PBS, and the resulting cells, together with the frozen section, were immobilized with 2% paraformaldehyde at room temperature for 60 minutes. The resultant was washed with PBS, was then treated with 1% Triton-X for 15 minutes, and was then blocked with 10% normal rabbit serum or normal goat serum at room temperature for 10 minutes. Thereafter, the resultant was reacted with a rabbit anti-human α-fetoprotein antibody (Abeam, 500 times diluted) or with goat anti-human α1-antitrypsin antibody (Abeam, 300 times diluted) at 4° C. overnight. Thereafter, the resultant was washed with PBS, and was then reacted with FITC-conjugated anti-rabbit IgG or FITC-conjugated anti-goat IgG used as a secondary antibody at room temperature for 1 hour. The reaction mixture was washed with PBS, and the nucleus was then stained with DAPI, followed by observation under a fluorescence microscope (FIG. 6 and FIG. 8). In FIG. 6, almost no AFP-positive cells were observed in the control group, but many AFP-positive cells were observed in the normal liver group and in the hepatitis liver group. In FIG. 8, as in the case of AFP, many AAT-positive cells were observed in the normal liver group and in the hepatitis liver group, in comparison to in the control group. The number of iPS cells, which expressed α-fetoprotein and α1-antitrypsin, was counted, and the ratio of such iPS cells to the nucleated cells was then calculated (FIG. 7 and FIG. 9), so that differentiation induction efficiency was analyzed. As shown in FIG. 7, a significantly higher ratio of AFP-positive cells was observed in the normal liver group and in the hepatitis liver Day 1 group, than in the control group. Moreover, as shown in FIG. 9, a significantly higher ratio of AAT-positive cells was observed in the normal liver group and in the hepatitis liver Day 1, 2 and 5 groups, than in the control group.

Furthermore, using an anti-human GFAP antibody or an anti-CNPase antibody as a primary antibody, the expression of a protein was observed under a fluorescence microscope. iPS cells cultured on various types of frozen sections were immobilized with 2% paraformaldehyde for 1 hour, and were then washed with PBS twice. Thereafter, the resulting cells were treated for 15 minutes with 1% TRITON X-100 (ICN Biomedical) that had been diluted with 0.1% BSA-containing PBS. The resulting cells were washed with PBS twice, and were then blocked with 10% normal goat serum for 1 hour. The resultant was washed with PBS twice, and was then reacted with a primary antibody diluted with 1% BSA-containing PBS at 4° C. overnight. The used primary antibody was Rabbit anti-Glial Fibrillary Acidic Protein antibody (SIGMA, 80 times diluted) or Rabbit anti-CNPase antibody (Abeam, 100 times diluted). The reaction mixture was washed with PBS three times, and was then reacted with a secondary antibody diluted with 1% BSA-containing PBS for 1 hour. For GFAP, Goat anti-rabbit IgG FITC (Wako, 40 times diluted) was used as a secondary antibody, and for CNPase, Goat anti-rabbit IgG Alexa (Invitergen, 100 times diluted) was used as a secondary antibody. The resultant was washed with PBS twice, and was then stained with DAPI for 30 minutes. The resultant was washed with PBS twice, was then enclosed with VECTASHIELD, and was then observed under a fluorescence microscope.

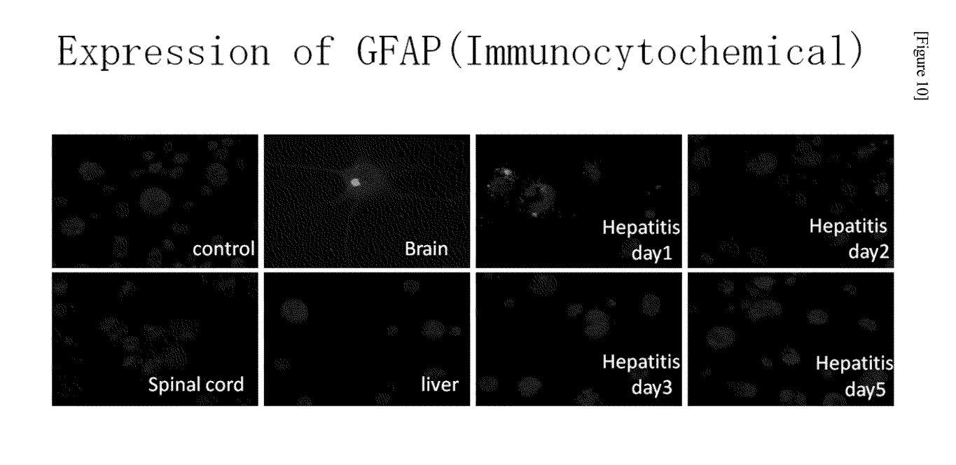

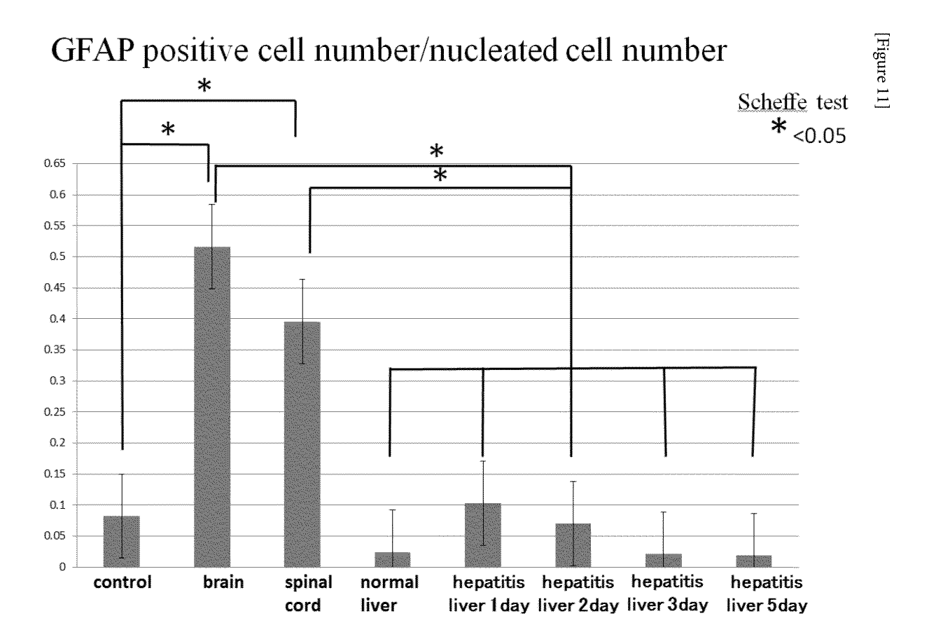

The results obtained by observing under a fluorescence microscope the expression of a protein using an anti-human GFAP antibody as a primary antibody are shown in FIG. 10. The number of iPS cells, which expressed GFAP, was counted, and the ratio of such iPS cells to the nucleated cells was then calculated (FIG. 11), so that differentiation induction efficiency was analyzed.

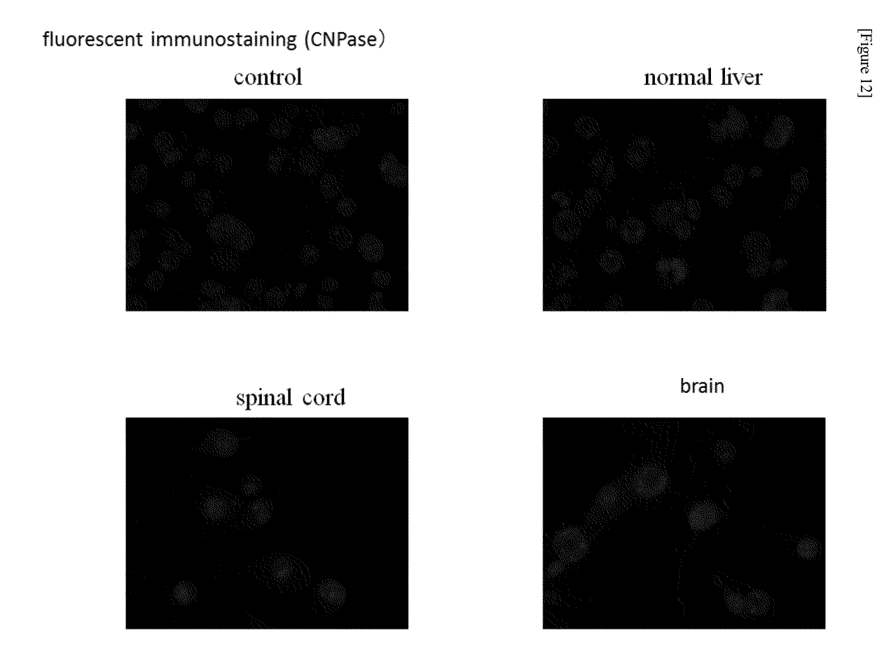

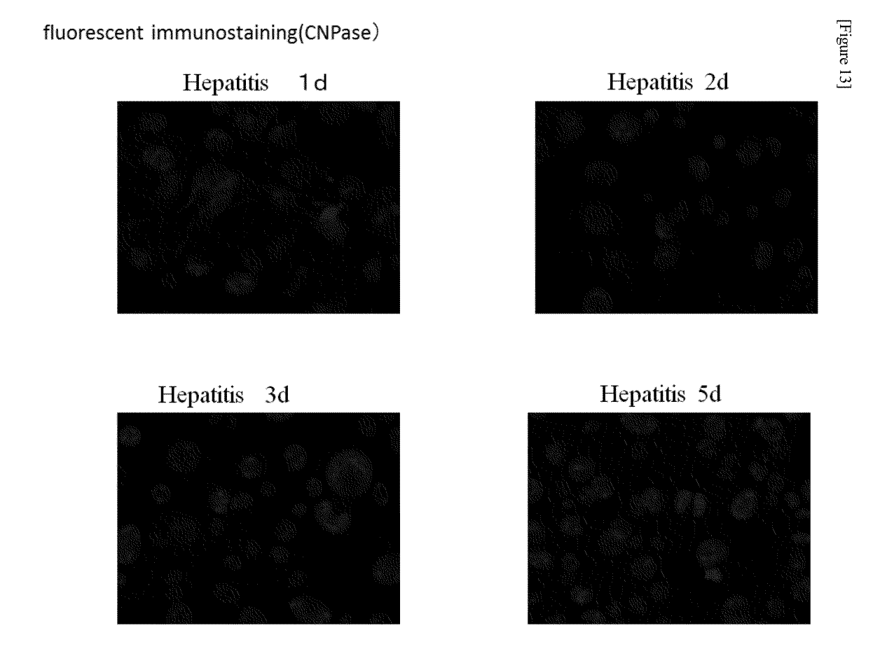

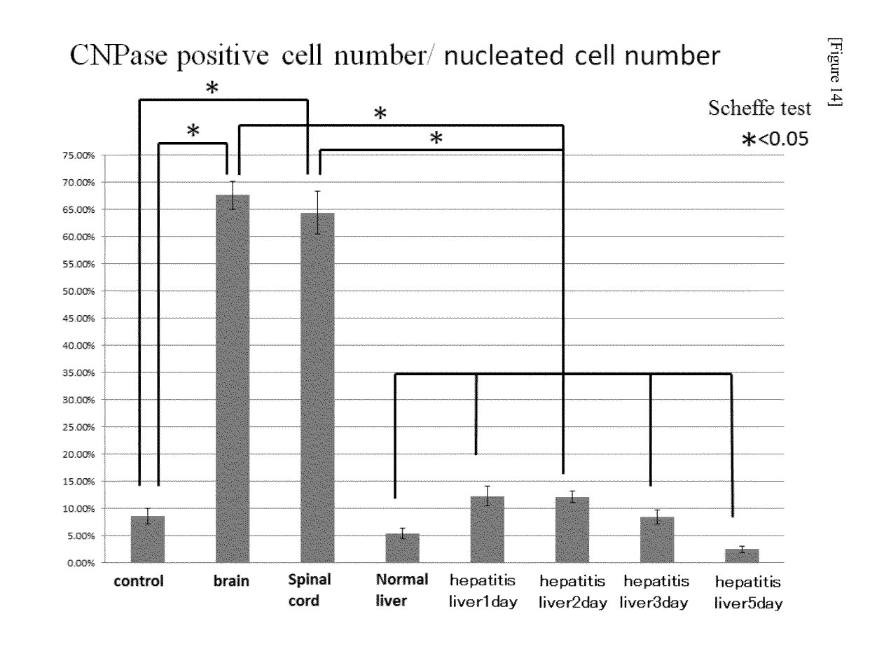

The results obtained by observing under a fluorescence microscope the expression of a protein using an anti-CNPase antibody as a primary antibody are shown in FIG. 12 and FIG. 13. The number of iPS cells that expressed CNPase was counted, and the ratio of such iPS cells to the nucleated cells was then calculated (FIG. 14), so that differentiation induction efficiency was analyzed.

The results regarding cell morphology and the expression of genes (AFP, AAT, and ALB) in a case where iPS cells were cultured on a cover glass (control), on a normal liver section, and on a hepatitis liver section are shown in Table 2. The results regarding the expression of the proteins (AFP and AAT) are shown in Table 3. In addition, the results regarding the expression of GFAP and CNPase genes and proteins are shown in Table 4.

TABLE 2

| | Cover glass | | |
|---|---|---|---|
| | (control) | Normal liver | Hepatitis liver |
| Cell | No uniformity found | Large polygonal | Large polygonal |
| morphology | | | |
| AFP | + | +++ | ++ |
| AAT | + | ++ | + |
| ALB | − | ++ | + |

TABLE 3

| | Cover | Nor- | Hepatitis | Hepatitis | Hepatitis | Hepatitis |
|---|---|---|---|---|---|---|
| | glass | mal | liver | liver | liver | liver |
| | (control) | liver | (Day 1) | (Day 2) | (Day 3) | (Day 5) |
| AFP | ± | +++ | +++ | ++ | + | ++ |
| AAT | ± | +++ | +++ | ++ | + | ++ |

TABLE 4

| | | | | | Hepatitis | Hepatitis | Hepatitis | Hepatitis |
|---|---|---|---|---|---|---|---|---|
| | | | Spinal | Normal | liver | liver | liver | liver |
| | Control | Brain | cord | liver | (Day 1) | (Day 2) | (Day 3) | (Day 5) |
| Cell | No | Asymmetric, | Asymmetric, | large | large | large | large | large |
| morphology | uniformity | with | with | polygonal | polygonal | polygonal | polygonal | polygonal |
| | found | projections | projection | | | | | |
| GFAP gene | + | ++ | ++ | − | − | − | − | − |
| expression | | | | | | | | |
| CNPase | + | +++ | ++ | − | − | − | − | − |
| gene | | | | | | | | |
| expression | | | | | | | | |
| GFAP | ± | + | + | ± | ± | ± | ± | ± |
| protein | | | | | | | | |
| expression | | | | | | | | |
| CNPase | ± | + | + | ± | ± | ± | ± | ± |
| protein | | | | | | | | |
| expression | | | | | | | | |

From the aforementioned results, it was demonstrated that the method of the present invention enables induction of the differentiation of iPS cells into hepatic cells and nerve cells.

1. A method for inducing differentiation of induced pluripotent stem cells, which comprises culturing induced pluripotent stem cells on a structure comprising cells and/or a component derived from the cells, wherein the cells in the structure and the differentiation-induced cells are the same type of cell.

2. The method according to claim 1, wherein the structure comprising cells and/or a component derived from the cells is a sheet-like structure.

3. The method according to claim 1, wherein the structure comprising cells and/or a component derived from the cells is a culture substrate coated with a component derived from biological tissues, organ sections or cells.

4. The method according to claim 1, wherein the cells are cells of the liver, brain or spinal cord.

5. A method for producing differentiation-induced cells, which comprises culturing induced pluripotent stem cells on a structure comprising cells and/or a component derived from the cells, wherein the cells in the structure and the differentiation-induced cells are the same type of cell.

6. The method according to claim 5, wherein the structure comprising cells and/or a component derived from the cells is a sheet-like structure.

7. The method according to claim 6, wherein the structure comprising cells and/or a component derived from the cells is a culture substrate coated with a component derived from biological tissues, organ sections or cells.

8. The method according to claim 5, wherein the cells are cells of the liver, brain or spinal cord.

9. A method for selecting induced pluripotent stem cells, which comprises culturing induced pluripotent stem cells on a structure comprising cells and/or a component derived from the cells, so as to induce differentiation of the induced pluripotent stem cells, and selecting induced pluripotent stem cells having high differentiation induction efficiency, wherein the cells in the structure and the differentiation-induced cells are the same type of cell.

10. The method according to claim 9, wherein the structure comprising cells and/or a component derived from the cells is a sheet-like structure.

11. The method according to claim 9, wherein the structure comprising cells and/or a component derived from the cells is a culture substrate coated with a component derived from biological tissues, organ sections or cells.

12. The method according to claim 9, wherein the cells are cells of the liver, brain or spinal cord.

13. A kit for selecting induced pluripotent stem cells, which comprises at least a structure comprising cells and/or a component derived from the cells.

14. The kit according to claim 13, wherein the structure comprising cells and/or a component derived from the cells is a sheet-like structure.

15. The kit according to claim 13, wherein the structure comprising cells and/or a component derived from the cells is a culture substrate coated with a component derived from biological tissues, organ sections or cells.

16. The kit according to claim 13, wherein the cells are cells of the liver, brain or spinal cord.
